SEQUENCE LISTING (11 sequences)

SEQ ID NO: 1 - DNA, 19 bp, Artificial Sequence
  Description of Artificial Sequence Synthetic oligonucleotide
ccagaaggccctattgatc

SEQ ID NO: 2 - DNA, 20 bp, Artificial Sequence
  Description of Artificial Sequence Synthetic oligonucleotide
gccagaaggccctattgatc

SEQ ID NO: 3 - DNA, 21 bp, Artificial Sequence
  Description of Artificial Sequence Synthetic oligonucleotide
gatcaatagggccttctggta

SEQ ID NO: 4 - DNA, 9 bp, Artificial Sequence
  Description of Artificial Sequence Synthetic oligonucleotide
ttcaagaga

SEQ ID NO: 5 - DNA, 66 bp, Artificial Sequence
  Description of Artificial Sequence Synthetic oligonucleotide
gatccgccagaaggccctattgatcttcaagagagatcaatagggccttctggtattttttggaaa

SEQ ID NO: 6 - DNA, 66 bp, Artificial Sequence
  Description of Artificial Sequence Synthetic oligonucleotide
agcttttccaaaaaataccagaaggccctattgatctctcttgaagatcaatagggccttctggcg

SEQ ID NO: 7 - DNA, 20 bp, Artificial Sequence
  Description of Artificial Sequence Synthetic primer
ggcgttggattatgctcatt

SEQ ID NO: 8 - DNA, 20 bp, Artificial Sequence
  Description of Artificial Sequence Synthetic primer
ccctgatcaatagggccttc

SEQ ID NO: 9 - DNA, 20 bp, Artificial Sequence
  Description of Artificial Sequence Synthetic primer
ccgatgctgctaagaacaca

SEQ ID NO: 10 - DNA, 20 bp, Artificial Sequence
  Description of Artificial Sequence Synthetic primer
cttcagttccagcccaactc

SEQ ID NO: 11 - DNA, 18 bp, Artificial Sequence
  Description of Artificial Sequence Synthetic primer
tttttttttttttttttt

(19) United States
(12) Patent Application Publication
(10) Pub. No.: US 2010/0028949 A1
(43) Pub. Date: Feb. 4, 2010

(54) SHRNA-MEDIATED INHIBITION OF EXPRESSION OF ALPHA 1,6-FUCOSYLTRANSFERASE

(76) Inventors: Vincent Beuger, Koeln (DE); Helmut Burtscher, Habach (DE); Christian Klein, Iffeldorf (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/518,128

(22) PCT Filed: Dec. 19, 2007
(86) PCT No.: PCT/EP07/11160

§ 371 Date: Jun. 8, 2009

(30) Foreign Application Priority Data
Dec. 22, 2006 (EP) 06026653.3

(51) Int. Cl. C12P 21/08; C12N 15/11

(52) U.S. Cl. 435/69.6; 536/23.5; 435/325

(57) ABSTRACT

The current invention comprises a method for producing a heterologous polypeptide with a reduced degree of fucose modification in a mammalian cell by cultivating the mammalian cell under conditions suitable for the expression of said heterologous polypeptide, and recovering the heterologous polypeptide from the mammalian cell or the culture, wherein in said mammalian cell the enzymatic activity of α1,6-fucosyltransferase is reduced by means of an shRNA directed against α1,6-fucosyltransferase mRNA.

The present invention relates to the field of RNAi. More precisely, the present invention relates to the field of reducing the translation of enzymes which catalyze modification of recombinantly produced proteins such as diagnostic or therapeutic antibodies.

### BACKGROUND OF THE INVENTION

The phenomenon of RNAi mediated gene silencing has been described first in the Caenorhabditis elegans system, in which microinjection of long double stranded RNA molecules was reported to result in an inactivation of the respective gene (U.S. Pat. No. 6,506,559). Later on, RNAi mediated gene silencing has been disclosed in vertebrates (EP 1 114 784), in mammals, and in particular in human cells (EP 1 144 623). In these systems, gene inactivation is achieved successfully, if short, double stranded RNA molecules of 19-29 bp are transfected in order to transiently knock down a specific gene of interest.

The mechanism of RNA mediated gene inactivation seems to be slightly different in the various organisms that have been investigated so far. In all systems, however, RNA mediated gene silencing is based on post-transcriptional degradation of the target mRNA induced by the endonuclease Argonaute2 which is part of the so called RISC complex (WO 03/93430). Sequence specificity of degradation is determined by the nucleotide sequence of the specific antisense RNA strand loaded into the RISC complex.

Appropriate possibilities of introduction include transfecting the double stranded RNA molecule itself or in vivo transcription of DNA vector constructs which directly result in a short double stranded RNA compound having a sequence that is identical to a part of the target RNA molecule. In many cases, so called shRNA constructs have been used successfully for gene silencing. These constructs encode a stem-loop RNA, characterized in that after introduction into cells, it is processed into a double stranded RNA compound, the sequence of which corresponds to the stem of the original RNA molecule.

IgG1-type immunoglobulins have two N-linked oligosaccharide chains bound to the Fc region at position Asn297 or in some cases at position Asn298. N-linked oligosaccharides generally are of the complex biantennary type, composed of a trimannosyl core structure with the presence or absence of core fucose (Rademacher, T. W., et al., Biochem. Soc. Symp. 51 (1986) 131-148; Umana, P., et al., Nature Biotechnol. 17 (1999) 176-180; Okazaki, A., et al., J. Mol. Biol. 336 (2004) 1239-1249; Shinkawa, T., et al., J. Biol. Chem. 278 (2003) 3466-3473).

US 2004/0132140 and US 2004/0110704 report recombinant or genetic methods in order to inhibit α1,6-fucosyltransferase within cell lines expressing recombinant antibodies.

### SUMMARY OF THE INVENTION

The present invention comprises a method for producing a heterologous polypeptide with a reduced degree of fucose modification in a mammalian cell comprising
cultivating the mammalian cell under conditions suitable for the expression of the heterologous polypeptide,
recovering the heterologous polypeptide from the mammalian cell or the culture,
   whereby the mammalian cell is transfected with a nucleic acid of SEQ ID NO: 5 or SEQ ID NO: 6, which is transcribed to a shRNA directed against α1,6-fucosyltransferase mRNA and with a nucleic acid encoding a heterologous polypeptide, preferably encoding an immunoglobulin, immunoglobulin fragment, or immunoglobulin conjugate as heterologous polypeptide.

In one embodiment, transcription of the shRNA is under control of a Pol III promoter, preferably of the U6 promoter. In one embodiment the mammalian cell is additionally transfected with a nucleic acid encoding a neomycin selection marker. In one embodiment the mammalian cell is a CHO derived cell. In one embodiment the mammalian cell is transfected with a single nucleic acid that comprises a first nucleic acid of SEQ ID NO: 5 or SEQ ID NO: 6 that is transcribed to an shRNA directed against α1,6-fucosyltransferase, a second nucleic acid encoding a neomycin selection marker, and a third nucleic acid encoding a heterologous polypeptide.

The present invention further comprises a nucleic acid comprising a first nucleic acid selected from the group of nucleic acids of SEQ ID NO: 5 and 6, a second nucleic acid encoding a neomycin selection marker, and a third nucleic acid encoding a heterologous polypeptide selected from the group of heterologous polypeptides comprising immunoglobulins, immunoglobulin fragments, and immunoglobulin conjugates.

The present invention also reports a cell comprising the nucleic acid according to the invention.

### DETAILED DESCRIPTION OF THE INVENTION

The present invention comprises a method for recombinantly producing a heterologous polypeptide with a reduced degree of fucose modification in a mammalian cell, which comprises a nucleic acid that is transcribed to an shRNA and a nucleic acid encoding the heterologous polypeptide, comprising transfecting the mammalian cell with said nucleic acid, cultivating the transfected mammalian cell under conditions suitable for the expression of the heterologous polypeptide, and recovering the heterologous polypeptide from the mammalian cell or the culture, whereby in the mammalian cell the enzymatic activity of α1,6-fucosyltransferase is reduced by means of the transcribed shRNA which is directed against α1,6-fucosyltransferase mRNA.

It has surprisingly been found that with a nucleic acid of SEQ ID NO: 5 or SEQ ID NO: 6, which is transcribed to an shRNA, an immunoglobulin, or immunoglobulin fragment, or immunoglobulin conjugate with a reduced degree of fucose modification compared to known methods can be obtained by the cultivation of a mammalian cell comprising said nucleic acid.

The present invention further comprises a nucleic acid comprising an (first) expression cassette for transcribing a shRNA against α1,6-fucosyltransferase selected from SEQ ID NO: 5 and 6, an (second) expression cassette for expressing a neomycin selection marker, and an (third) expression cassette for expressing a heterologous polypeptide.

The present invention further comprises a mammalian cell comprising the nucleic acid according to the invention.

Methods and techniques known to a person skilled in the art, which are useful for carrying out the current invention, are described e.g. in Ausubel, F. M., ed., Current Protocols in Molecular Biology, Volumes I to III (1997); Sambrook et al., Molecular Cloning: A Laboratory Manual, Second Edition, Cold Spring Harbor Laboratory Press, Cold Spring Harbor, N.Y. (1989); Glover, N. D., and Hames, B. D., ed., DNA Cloning: A Practical Approach, Volumes I and II (1995), Oxford University Press; Freshney, R. I. (ed.), Animal Cell Culture—a practical approach, IRL Press (1986); Watson, J. D., et al., Recombinant DNA, Second Edition, CHSL Press (1992); Winnacker, E. L., From Genes to Clones, N. Y., VCH Publishers (1987); Celis, J., ed., Cell Biology, Second Edition, Academic Press (1998); Freshney, R. I., Culture of Animal Cells: A Manual of Basic Techniques, Second Edition, Alan R. Liss, Inc., N.Y. (1987).

The use of recombinant DNA technology enables the production of numerous derivatives of a nucleic acid and/or polypeptide. Such derivatives can, for example, be modified in one individual or several positions by substitution, alteration, exchange, deletion, or insertion. The modification or derivatisation can, for example, be carried out by means of site directed mutagenesis. Such modifications can easily be carried out by a person skilled in the art (see e.g. Sambrook, J., et al., Molecular Cloning: A laboratory manual (1989) Cold Spring Harbor Laboratory Press, New York, USA; Hames, B. D., and Higgins, S. G., Nucleic acid hybridization—a practical approach (1985) IRL Press, Oxford, England).

The use of recombinant technology enables the transformation of various host cells with one or more heterologous nucleic acid(s). Although the transcription and translation, i.e. expression, machinery of different cells use the same elements, cells belonging to different species may have among other things a different so-called codon usage. Thereby identical polypeptides (with respect to amino acid sequence) may be encoded by different nucleic acid(s). Also, due to the degeneracy of the genetic code, different nucleic acids may encode the same polypeptide.

A “nucleic acid” as used herein, refers to a polynucleotide molecule, for example to DNA, RNA, or modifications thereof. This polynucleotide molecule can be a naturally occurring polynucleotide molecule or a synthetic polynucleotide molecule or a combination of one or more naturally occurring polynucleotide molecules with one or more synthetic polynucleotide molecules. Also encompassed by this definition are naturally occurring polynucleotide molecules in which one or more nucleotides are changed, e.g. by mutagenesis, deleted, or added. A nucleic acid can either be isolated, or integrated in another nucleic acid, e.g. in an expression cassette, a plasmid, or the chromosome of a host cell. A nucleic acid is likewise characterized by its nucleic acid sequence consisting of individual nucleotides.

To a person skilled in the art procedures and methods are well known to convert an amino acid sequence of, e.g., a polypeptide into a corresponding nucleic acid sequence encoding the amino acid sequence. Therefore, a nucleic acid is characterized by its nucleic acid sequence consisting of individual nucleotides and likewise by the amino acid sequence of a polypeptide encoded thereby.

The term “plasmid” includes e.g. shuttle and expression plasmids/vectors as well as transfection plasmids/vectors. The terms “plasmid” and “vector” are used interchangeably within this application. Typically, a “plasmid” will also comprise an origin of replication (e.g. the ColE1 or oriP origin of replication) and a selection marker (e.g. an ampicillin, kanamycin, tetracycline, or chloramphenicol selection marker), for replication and selection, respectively, of the vector/plasmid in bacteria.

An “expression cassette” refers to a construct that contains the necessary regulatory elements, such as promoter and polyadenylation site, for expression of at least the contained nucleic acid, e.g. of a structural gene, in a cell. Optionally additional elements may be contained which e.g. enable the secretion of the expressed polypeptide. It is also within the scope of the invention to use the term expression cassette if the contained nucleic acid is after transcription not further translated into a polypeptide but forms, e.g., an shRNA.

A “structural gene” denotes the coding region of a gene without a signal sequence.

A “gene” denotes a nucleic acid segment, e.g. on a chromosome or on a plasmid, which is necessary for the expression of a polypeptide or protein. Beside the coding region the gene comprises other functional elements including promoters, introns, terminators, and optionally a leader peptide.

A “selection marker” is a nucleic acid that allows cells carrying the selection marker to be specifically selected for or against, in the presence of a corresponding selection agent. A useful positive selection marker is an antibiotic resistance gene. This selection marker allows the host cell transformed therewith to be positively selected for in the presence of the corresponding selection agent, e.g. the antibiotic. A non-transformed host cell is not capable to grow or survive under the selective conditions in the culture. A selection marker can be positive, negative, or bifunctional. Positive selection markers allow selection for cells carrying the marker, whereas negative selection markers allow cells carrying the marker to be selectively eliminated. Typically, a selection marker will confer resistance to a drug or compensate for a metabolic or catabolic defect in the host cell. Selection markers used with eukaryotic cells include, e.g., the genes for aminoglycoside phosphotransferase (APH), such as e.g. the hygromycin (hyg), neomycin (neo), and G418 selection markers, dihydrofolate reductase (DHFR), thymidine kinase (tk), glutamine synthetase (GS), asparagine synthetase, tryptophan synthetase (selection agent indole), histidinol dehydrogenase (selection agent histidinol D), and nucleic acids conferring resistance to puromycin, bleomycin, phleomycin, chloramphenicol, Zeocin, and mycophenolic acid. Further marker genes are described e.g. in WO 92/08796 and WO 94/28143.

The term “expression” as used herein refers to transcription and/or translation processes occurring within a cell. The level of transcription of a desired product in a host cell can be determined on the basis of the amount of corresponding mRNA that is present in the cell. For example, mRNA transcribed from a sequence of interest can be quantitated by PCR or by Northern hybridization (see Sambrook et al., Molecular Cloning: A Laboratory Manual, Cold Spring Harbor Laboratory Press (1989)). Polypeptides encoded by a nucleic acid of interest can be quantitated by various methods, e.g. by ELISA, by assaying for the biological activity of the polypeptide, or by employing assays that are independent of such activity, such as Western blotting or radioimmunoassay, using immunoglobulins that recognize and bind to the polypeptide (see Sambrook et al., 1989, supra).

The term “under conditions suitable for the expression of the heterologous polypeptide” denotes conditions which are used for the cultivation of a mammalian cell in order to express a heterologous polypeptide, which is encoded by a nucleic acid which has been transfected into said mammalian cell, and which are known to or can easily be determined by a person skilled in the art. It is also known to a person skilled in the art that these conditions may vary depending on the type of mammalian cell cultivated and type of protein expressed. In general the mammalian cell is cultivated at a temperature, e.g. between 20° C. and 40° C., and for a period of time sufficient to allow effective protein production, e.g. for 4 to 28 days.

The term “cell” or “host cell” refers to a cell into which a nucleic acid, e.g. encoding a heterologous polypeptide or constituting a shRNA, can be or is introduced/transfected. Host cells include both prokaryotic cells, which are used for propagation of vectors/plasmids, and eukaryotic cells, which are used for the expression of the nucleic acid. Preferably, the eukaryotic cells are mammalian cells. Preferably the mammalian (host) cell is selected from the mammalian cells like CHO cells (e.g. CHO K1 or CHO DG44), BHK cells, NS0 cells, SP2/0 cells, HEK 293 cells, HEK 293 EBNA cells, PER.C6 cells, or COS cells. Preferably the mammalian cell is selected from the group comprising hybridoma, myeloma, and rodent cells. Myeloma cells comprise rat myeloma cells (e.g. YB2), and mouse myeloma cells (e.g. NS0, SP2/0).

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is produced, and may vary with the type of cell. Polypeptides are defined herein in terms of their amino acid backbone structure. Additions such as carbohydrate groups are generally not specified, but may be present nonetheless.

The term “amino acid” as used within this application denotes a group of carboxy α-amino acids, which directly or in form of a precursor can be encoded by a nucleic acid, comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

As used herein, the term “immunoglobulin” denotes a protein consisting of one or more polypeptides substantially encoded by immunoglobulin genes. This definition includes variants such as mutated forms, i.e. forms with substitutions, deletions, and insertions of one or more amino acids, truncated forms, fused forms, chimeric forms, as well as humanized forms. The recognized immunoglobulin genes include the different constant region genes as well as the myriad immunoglobulin variable region genes from, e.g., primates and rodents. Immunoglobulins may exist in a variety of formats, including, for example, Fv, Fab, and (Fab)₂, as well as single chains (scFv) (e.g. Huston, J. S., et al., Proc. Natal. Acad. Sci. USA 85 (1988) 5879-5883; Bird, R. E., et al., Science 242 (1988) 423-426; and, in general, Hood et al., Immunology, Benjamin N.Y., 2nd edition (1984) and Hunkapiller, T., and Hood, L., Nature 323 (1986) 15-16). Monoclonal immunoglobulins are preferred.

Each of the heavy and light polypeptide chains of an immunoglobulin, if present at all, may comprise a constant region (generally the carboxyl terminal portion). Each of the heavy and light polypeptide chains of an immunoglobulin, if present at all, may comprise a variable domain (generally the amino terminal portion). The variable domain of an immunoglobulin's light or heavy chain may comprise different regions, i.e. four framework regions (FR) and three hypervariable regions (CDR).

The term “monoclonal immunoglobulin” as used herein refers to an immunoglobulin obtained from a population of substantially homogeneous immunoglobulins, i.e. the individual immunoglobulins comprising the population are identical except for possible naturally occurring mutations that may be present in minor amounts. Monoclonal immunoglobulins are highly specific, being directed against a single antigenic site (epitope). Furthermore, in contrast to polyclonal immunoglobulin preparations, which include different immunoglobulins directed against different antigenic sites (determinants or epitopes), each monoclonal immunoglobulin is directed against a single antigenic site on the antigen. In addition to their specificity, the monoclonal immunoglobulins are advantageous in that they may be synthesized uncontaminated by other immunoglobulins. The modifier “monoclonal” indicates the character of the immunoglobulin as being obtained from a substantially homogeneous population of immunoglobulins and is not to be construed as requiring production of the immunoglobulin by any particular method.

“Humanized” forms of non-human (e.g. rodent) immunoglobulins are chimeric immunoglobulins that contain partial sequences derived from non-human immunoglobulin and from human immunoglobulin. For the most part, humanized immunoglobulins are derived from a human immunoglobulin (recipient immunoglobulin), in which residues from a hypervariable region are replaced by residues from a hypervariable region of a non-human species (donor immunoglobulin), such as mouse, rat, rabbit, or non-human primate, having the desired specificity and affinity (see e.g. Morrison, S. L., et al., Proc. Natal. Acad. Sci. USA 81 (1984) 6851-6855; U.S. Pat. No. 5,202,238; U.S. Pat. No. 5,204,244). In some instances, framework region (FR) residues of the human immunoglobulin are replaced by corresponding non-human residues. Furthermore, humanized immunoglobulins may comprise further modifications, e.g. amino acid residues that are not found in the recipient immunoglobulin or in the donor immunoglobulin. Such modifications result in variants of such recipient or donor immunoglobulin, which are homologous but not identical to the corresponding parent sequence. These modifications are made to further refine immunoglobulin performance.

In general, the humanized immunoglobulin will comprise substantially all of at least one, and typically two, variable domains, in which all or substantially all of the hypervariable loops correspond to those of a non-human donor immunoglobulin and all or substantially all of the FRs are those of a human recipient immunoglobulin. The humanized immunoglobulin optionally will also comprise at least a portion of an immunoglobulin constant region, typically that of a human immunoglobulin.

Methods for humanizing non-human immunoglobulin have been described in the art. Preferably, a humanized immunoglobulin has one or more amino acid residues introduced into it from a source which is non-human. These non-human amino acid residues are often referred to as “import” residues, which are typically taken from an “import” variable domain. Humanization can be essentially performed following the method of Winter and co-workers (Jones, P. T., et al., Nature 321 (1986) 522-525; Riechmann, L., et al., Nature 332 (1988) 323-327; Verhoeyen, M., et al., Science 239 (1988) 1534-1536; Presta, L. G., Curr. Op. Struct. Biol. 2 (1992) 593-596), by substituting hypervariable region sequences for the corresponding sequences of a human immunoglobulin. Accordingly, such “humanized” immunoglobulins are chimeric immunoglobulins (see e.g. U.S. Pat. No. 4,816,567), wherein substantially less than an intact human variable domain has been substituted by the corresponding sequence from a non-human species. In practice, humanized immunoglobulins are typically human immunoglobulins in which some hypervariable region residues and possibly some framework region residues are substituted by residues from analogous sites in rodent or non-human primate immunoglobulins.

Recombinant production of immunoglobulins is well-known in the state of the art and reported, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-160; Werner, R. G., Drug Research 48 (1998) 870-880.

Preferably the heterologous polypeptide is selected from the group comprising immunoglobulins, immunoglobulin fragments, immunoglobulin conjugates. Preferably said immunoglobulin, immunoglobulin fragment, or immunoglobulin conjugate is a monoclonal immunoglobulin, a monoclonal immunoglobulin fragment, or a monoclonal immunoglobulin conjugate.

As used herein the term “immunoglobulin fragment” denotes a part of an immunoglobulin. Immunoglobulin fragments comprise Fv, Fab, (Fab)₂, single chains (scFv), as well as single heavy chains and single light chains, as well as immunoglobulins in which at least one region and/or domain selected from the group comprising framework region 1, framework region 2, framework region 3, framework region 4, hypervariable region 1, hypervariable region 2, hypervariable region 3, each of a light and heavy chain, Fab-region, hinge-region, variable region, heavy chain constant domain 1, heavy chain constant domain 2, heavy chain constant domain 3, and light chain constant domain, has been deleted.

As used herein the term “immunoglobulin conjugate” denotes a fusion of an immunoglobulin and a polypeptide. The term immunoglobulin conjugate comprises fusion proteins of an immunoglobulin or an immunoglobulin fragment with one to eight, preferably two or four, polypeptides, whereby each of the polypeptides is fused to a different N- or C-terminal amino acid with or without an intervening linker polypeptide. If the immunoglobulin conjugate comprises more than one non-immunoglobulin polypeptide, each of the conjugated non-immunoglobulin polypeptides can have the same or a different amino acid sequence and/or length.

As used herein, the expression “cell” includes the subject cell and its progeny. Thus, the words “transformant” and “transformed cell” include the primary subject cell and cultures derived there from without regard for the number of transfers. It is also understood that all progeny may not be precisely identical in DNA content, due to deliberate or inadvertent mutations. Variant progeny that have the same function or biological activity as screened for in the originally transformed cell are included.

The heterologous polypeptide according to the invention is produced by recombinant means. Such methods are widely known in the state of the art and comprise protein expression in prokaryotic and eukaryotic cells with subsequent recovery and isolation of the heterologous polypeptide and usually purification to a pharmaceutically acceptable purity. In case of the heterologous polypeptide being an immunoglobulin, nucleic acids encoding light and heavy chains or fragments thereof or conjugates thereof are inserted into expression cassettes by standard methods. Nucleic acids encoding immunoglobulins are readily isolated and sequenced using conventional procedures. Hybridoma cells can serve as a source of such nucleic acid. The expression cassettes may be inserted into one or more expression vectors, which are then transfected into a (host) cell, which do not otherwise produce immunoglobulins. Expression is performed in appropriate eukaryotic (host) cells and the immunoglobulin is recovered from the cells after lysis or from the supernatant.

Recombinant production of antibodies is well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-160; Werner, R. G., Drug Research 48 (1998) 870-880.

Different methods are well established and widespread used for protein recovery and purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A., Appl. Biochem. Biotech. 75 (1998) 93-102).

The present invention is applicable in general in all living cells expressing the so-called double-strand RNA nuclease Dicer and the RISC complex or, in other words in all cells where RNA mediated gene silencing can be observed. Thus, the present invention can be applied predominantly for mammalian cell lines, but also for all types of eukaryotic cells. Preferred however, are cell lines which are commonly used for producing recombinant polypeptides such as for example Chinese Hamster Ovary cells, e.g. CHO K1 (Jones, C., et al., Cytogenet. Cell Genet. 16 (1976) 387-390), or CHO DG44 (Urlaub, G, et al., Cell 33 (1983) 405-412; Urlaub, G., et al., Somat. Cell. Mol. Genet. 12 (1986) 555-566), Human Embryonic Kidney cells, such as e.g. HEK293 cells (Graham, F. L., et al., J. Gen. Virol. 36 (1977) 59-74), or HEK293 EBNA cells, NS0 cells (Barnes L. M., et al., Cytotechnology 32 (2000) 109-123; Barnes, L. M., et al., Biotech. Bioeng. 73 (2001) 261-270), and/or SP2/0 cells (Shulman, M., et al., Nature 276 (1978) 269-270).

In the context of the present invention, the term “reduction of enzymatic activity of α1,6-fucosyltransferase” and grammatical equivalents thereof denote the degradation of the specific target mRNA encoding said α1,6-fucosyltransferase in cells used for the expression of heterologous polypeptides, which is mediated by a shRNA compound. The shRNA compound itself is synthesized after transfection of the (host) cell with an appropriate expression cassette constituting said shRNA compound. Alternatively transfection with a precursor of an RNAi compound, which is subsequently processed into an RNAi compound, is possible.

The RNAi compound according to the present invention is a shRNA directed against the mRNA encoding α1,6-fucosyltransferase (targeted mRNA). So far, two major gene silencing strategies have emerged for in vitro studies: small interfering RNAs (siRNAs) and small hairpin RNAs (shRNAs) (Tuschl, T., Nature Biotechnol. 20 (2002) 446-448). Plasmid-derived shRNAs according to the present invention provide the option for combination with reporter genes or selection markers, and delivery via viral vectors (Brummelkamp, T. R., and Bernards, R., Nat. Rev. Cancer 3 (2003) 781-789). The transfection of cells with an RNAi compound results in cells having a reduced level of the target mRNA and, thus, of the corresponding polypeptide and, concurrently, of the corresponding enzyme activity. The mRNA level is of from 5% to 20%, preferably of from 5% to 15%, more preferably of from 5% to 10% of the mRNA level of the corresponding wild type cell. The wild type cell is the cell prior to the introduction of the nucleic acid encoding the RNAi compound, in which the targeted mRNA is not degraded by an RNAi compound.

Generation of stable cell clones is often a tedious and lengthy process. Thus, in one embodiment selection with a recombinantly expressed cell surface marker is used for the isolation of transfectants. It is within the scope of the present invention to use any kind of gene whose expression product is located on the cell surface as a marker for enrichment and selection of transfectants expressing a high level of shRNA compound. 1-NGFR, a truncated form of the low-affinity nerve growth factor receptor, and thus inactive for signal transduction, is expressed on the cell surface and has proven to be a highly useful marker for cell biological analysis (Phillips, K., et al., Nat. Med. 2 (1996) 1154-1156 and Machl, A. W., et al., Cytometry 29 (1997) 371-374).

Within the scope of the present invention, cell transformants may be obtained with substantially any kind of transfection method known in the art. For example, the vector DNA may be introduced into the cells by means of electroporation or microinjection. Alternatively, lipofection reagents such as FuGENE 6 (Roche Diagnostics GmbH), X-tremeGENE (Roche Diagnostics GmbH), LipofectAmine (Invitrogen Corp.) or nucleofection (AMAXA AG, cologne, Germany) may be used. Still alternatively, the vector DNA comprising expression cassettes for a cell surface protein and an shRNA compound may be introduced into the cell by appropriate viral vector systems based on retroviruses, lentiviruses, adenoviruses, or adeno-associated viruses (Singer, O., Proc. Natl. Acad. Sci. USA 101 (2004) 5313-5314).

In one embodiment the mammalian cell is transfected with a nucleic acid encoding a selection marker. Preferably the selection marker is selected from hygromycin, puromycin, and/or neomycin selection marker. In this embodiment selective pressure, i.e. the cultivation in the presence of a selection agent, results in the selection/growth of stably transfected cell lines. In one embodiment the selective pressure is by the addition of Lens culinaris agglutinin (LCA).

In one embodiment the invention comprises a method for recombinantly producing a heterologous polypeptide with a reduced degree of fucose modification in a mammalian cell comprising
cultivating the mammalian cell under conditions suitable for the expression of the heterologous polypeptide,
recovering the heterologous polypeptide from the mammalian cell or the culture,
   whereby the mammalian cell is transfected with a nucleic acid comprising a first nucleic acid of SEQ ID NO: 5 or of SEQ ID NO: 6 that is transcribed to an shRNA directed against α1,6-fucosyltransferase mRNA, a third nucleic acid encoding a neomycin selection marker, and a second nucleic acid encoding a heterologous polypeptide. In one embodiment is the mammalian cell transfected with a single nucleic acid. The term “single nucleic acid” denotes within this application a mixture of nucleic acids having the identical nucleic acid sequence despite single nucleotide changes emerging from the generation and production of the nucleic acid, wherein these changes have no effect on the encoded mRNAs. The term “identical nucleic acid sequence” denotes within this application that the nucleic acids used for transfecting said mammalian cell have an nucleotide identity of at least 90%, or at least 95%, or at least 98%, or of 98% or more.

The transcript derived from the nucleic acid which is constituting the shRNA compound can be either transcribed from Pol II promoters such as the CMV promoter or from a Pol III promoter like the H1, U6, or 7SK promoters (Zhou, H., et al., Nucleic Acids Res. 33 (2005) e62; Brummelkamp, T. R. and Bernards, R., Nat. Rev. Cancer 3 (2003) 781-789; Czauderna, F., et al., Nucleic Acids Res. 31 (2003) e127).

In case of a Pol III mediated transcription, it is essential to have a Pol III terminator sequence of TTTT, preferably a TTTTTT, at the 3′ end of the transcribed RNA for appropriate 3′ processing of the precursor RNA product (Dykxhoorn, D. M., et al., Nat. Rev. Mol. Cell Biol. 4 (2003) 457-467).

The RNAi compound is a RNA with a hairpin confirmation, i.e. an shRNA. As an active RNAi compound, such a molecule may start with a G nucleotide at its 5′ end, due to the fact that transcription from the H1 and U6 promoter usually starts with a G. The stem of the molecule is due to inverted repeat sequences and is 19 to 29, preferably 19 to 23, base pairs in length. Preferably, these inverted repeat sequences are completely complementary to each other and can form a double stranded hybrid without any internal mismatches.

The internal loop of the molecule is a single stranded chain of 4 to 40, preferably 4 to 9 nucleotides. For this loop, it is important to avoid any inverted repeat sequences in order to prevent the molecule from folding itself into an alternate secondary structure that is not capable of acting as an shRNA molecule.

At the 3′ end of the shRNA, there may be an overhang. In case of usage of a Pol III promoter, the overhang may be 2 to 4 U residues due to the terminator signal of Pol III promoters. When expressed within a cell, these hairpin constructs are rapidly processed into active double stranded molecules capable of mediating gene silencing (Dykxhoorn, D. M., et al., Nat. Rev. Mol. Cell Biol. 4 (2003) 457-467).

Nucleic acids (DNA) are composed of four nucleobases or nucleotide bases, A, C, T, and G. A denotes adenosine, C denotes cytidine, T denotes thymidine, and G denotes guanosine. In RNA thymidine is replaced by uridine (U).

The shRNA compound directed against the α1,6-fucosyltransferase mRNA is transcribed from an appropriate expression cassette. It comprises a stem of 19 to 29 nucleotides, preferably of 19 to 23 nucleotides, in length, whose sequence is identical/complementary to the target mRNA that has to be inactivated.

In one embodiment, the nucleic acid of the stem of the shRNA directed against α1,6-fucosyltransferase mRNA is selected from the group of nucleic acids comprising SEQ ID NO: 1 (CCAGAAGGCCCTATTGATC), SEQ ID NO: 2 (GCCAGAAGGCCCTATTGATC), and SEQ ID NO: 3 (GATCAATAGGGCCTTCTGGTA).

In one embodiment, the nucleic acid of the loop of the shRNA directed against α1,6-fucosyltransferase mRNA is the nucleic acid TTCAAGAGA (SEQ ID NO: 4).

In one embodiment the nucleic acid that is transcribed to an shRNA is selected from the group of nucleic acids comprising SEQ ID NO: 5 and 6, i.e. the nucleic acid that is transcribed to an shRNA has either the nucleic acid sequence of SEQ ID NO: 5, or the nucleic acid sequence of SEQ ID NO: 6.

With the method according to the invention a reduction of the target mRNA by about a factor of 50 can be achieved. Such a degree of reduction is enough to produce heterologous polypeptides with a reduced degree of fucosylation in a reasonably high yield.

The term “heterologous polypeptide with a reduced degree of fucose modification” and grammatical equivalents thereof denote a heterologous polypeptide, which is expressed in a mammalian cell, which has been transfected with a nucleic acid that is transcribed to an shRNA directed against α1,6-fucosyltransferase mRNA, and with a nucleic acid encoding the heterologous polypeptide, and whose fucosylation at the 6-position of an asparagine-linked N-acetylglucosamine is reduced in comparison with a heterologous polypeptide expressed in a mammalian cell of the same type, which is transfected with a nucleic acid encoding the heterologous polypeptide but not transfected with a nucleic acid transcribed to an shRNA directed against α1,6-fucosyltransferase mRNA. In one embodiment the ratio of the fucosylation of the heterologous polypeptide, which is expressed in a mammalian cell, which is transfected with a nucleic acid transcribed into an shRNA directed against α1,6-fucosyltransferase mRNA, and with a nucleic acid encoding the heterologous polypeptide, to the fucosylation of the heterologous polypeptide expressed in a mammalian cell of the same type, which is transfected with a nucleic acid encoding the heterologous polypeptide but not with a nucleic acid transcribed into an shRNA directed against α1,6-fucosyltransferase mRNA, is 15% or less. This denotes that the heterologous polypeptide is fucosylated to 15% or less. Preferably the ratio of the non-fucosylated heterologous polypeptide to the fucosylated heterologous polypeptide is 0.15 or less, i.e. for example 0.12.

“Heterologous DNA” or “heterologous polypeptide” refers to a DNA molecule or a polypeptide, or a population of DNA molecules, or a population of polypeptides, that do not exist naturally within a given host cell. DNA molecules heterologous to a particular host cell may contain DNA derived from the host cell species (i.e. endogenous DNA) so long as that host DNA is combined with non-host DNA (i.e. exogenous DNA). For example, a DNA molecule containing a non-host DNA segment encoding a polypeptide operably linked to a host DNA segment comprising a promoter is considered to be a heterologous DNA molecule. Conversely, a heterologous DNA molecule can comprise an endogenous structural gene operably linked with an exogenous promoter. A polypeptide encoded by a non-host DNA molecule is a “heterologous” polypeptide.

“Operably linked” refers to a juxtaposition of two or more components, wherein the components so described are in a relationship permitting them to function in their intended manner. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as a secretory leader/signal sequence and a polypeptide, contiguous and in reading frame. A polyadenylation site is operably linked to a coding sequence if it is located at the downstream end of the coding sequence such that transcription proceeds through the coding sequence into the polyadenylation sequence. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

The following examples, sequence listing and figures are provided to aid the understanding of the present invention, the true scope of which is set forth in the appended claims. It is understood that modifications can be made in the procedures set forth without departing from the spirit of the invention.

### BRIEF DESCRIPTION OF FIGURES

FIG. 1: Vector according to the invention for the transcription of shRNAFuT8

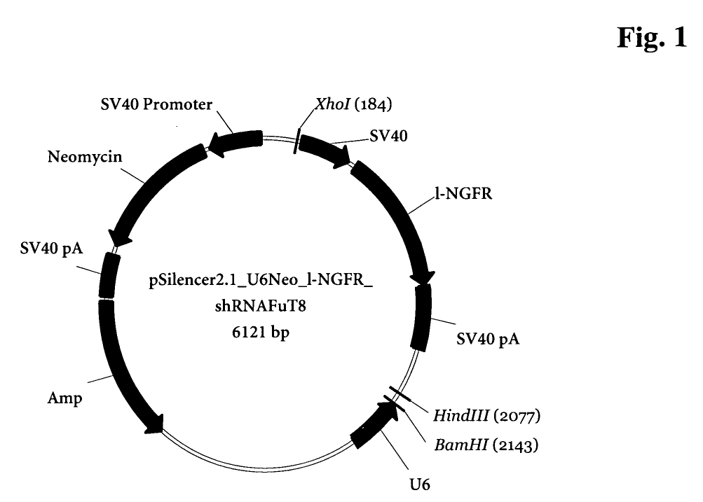

FIG. 2: Mass spectra indicating high (upper panel) and low (lower panel) amounts of differently fucosylated antibodies isolated from CHO cells transfected with shRNAFuT8 and subsequent selection with neomycin, 1-NGFR enrichment, and LCA-selection (upper panel) or only with neomycin selection as disclosed in Example 4.

FIG. 3: Schematic presentation of carbohydrate structures attached to an asparagine of antibody (GlcNAc=N-acetylglucosamine, Man=mannose, Gal=galactose, Fuc=fucose, NeuAc=N-acetyl neuraminic acid)

### EXAMPLES

### Example 1

### Vector Cloning

At position 184 of the vector pSilencer2.1_U6neo (Ambion Inc., cat. no. 5764) an XhoI site was introduced by site directed mutagenesis. An 1-NGFR (low affinity nerve growth factor; see e.g. Phillips, K., et al., Nat. Med. 2 (1996) 1154-1156 and Machl, A. W., et al., Cytometry 29 (1997) 371-374) expression cassette was subsequently cloned into the XhoI/HindIII restriction sites. In order to generate the FuT8 shRNA, the following oligonucleotides were employed:

F8shRNA4top

(SEQ ID NO: 5)

GATCCGCCAGAAGGCCCTATTGATCTTCAAGAGAGATCAATAGGG
CCTTCTGGTATTTTTTGGAAA
F8shRNA4bot

(SEQ ID NO: 6)

AGCTTTTCCAAAAAATACCAGAAGGCCCTATTGATCTCTCTTGAA
GATCAATAGGGCCTTCTGGCG

The annealed FuT8 shRNA was ligated into the corresponding vector fragment (BamHI/HindIII digested). The completed vector was called pSilencer2.1_U6neo_{—}1-NGFR_shRNAFuT8 (pSilencer).

### Example 2

### Selection and Isolation of Single Clones

CHO-DG44 cells were transfected with an antibody expressing plasmid. As exemplary antibody an antibody binding to the human insulin like growth factor receptor 1 was used (for sequences see e.g. WO 2005/005635).

The antibody producing CHO-DG44 clone (wild-type, without pSilencer) was transfected with pSilencer2.1_U6neo_{—}1-NGFR_shRNAFuT8 using FuGENE reagent (Roche Diagnostics GmbH) according to the manufacturer's manual. The stably transfected cells were cultured in MEM Alpha Medium (cat. no. 22561-021; Gibco®, Invitrogen GmbH, Germany) supplemented with 1% 200 mM L-Glutamine (Gibco) and 10% dialyzed gamma irradiated Fetal Bovine Serum (cat. no. 1060-017; Gibco®, Invitrogen GmbH, Germany). Transfected cells were selected with 400 μg/ml neomycin for one week. Surviving cells were 1-NGFR-enriched using the MACSelect-1-NGFR system according to the producer's manual (Miltenyi Biotec; Cat. 130-091-879). 1-NGFR-enriched cells were selected with 0.5 mg/ml LCA (Lens culinaris agglutinin). Clones of LCA-selected cells were recovered by diluting the LCA-selected pool to one cell per 96 well.

### Example 3

### RNA Isolation and cDNA Synthesis and Quantitative RT-PCR

Total RNA was isolated using the RNeasy Mini Kit (Qiagen GmbH, Germany) including DNAse digestion. Equal amounts of total RNA (400 ng) were reverse transcribed using the Transcriptor First Strand cDNA Synthesis Kit (Roche Diagnostics GmbH, Germany) with anchored oligo (dT)₁₈ primers. Samples were analyzed by real-time PCR after cDNA synthesis using the LightCycler FastStart DNA Master SYBR Green I kit (Roche Diagnostics GmbH, Germany). For amplification and detection of FuT8 (Fucosyltransferase 8) and ALAS (5-aminolaevulinate synthase) cDNA, sequence-specific primers were used as follows:

(SEQ ID NO: 7)

| | |
|---|---|
| FuT8 forward: | 5′-GGCGTTGGATTATGCTCATT-3′ |

(SEQ ID NO: 8)

| | |
|---|---|
| FUT8 reverse: | 5′-CCCTGATCAATAGGGCCTTC-3′ |

(SEQ ID NO: 9)

| | |
|---|---|
| ALAS forward: | 5′-CCGATGCTGCTAAGAACACA-3′ |

(SEQ ID NO: 10)

| | |
|---|---|
| ALAS reverse: | 5′-CTTCAGTTCCAGCCCAACTC-3′ |

Amplification was performed under the following conditions: a 10-minutes pre-incubation step at 95° C., followed by 45 cycles of 10 seconds at 95° C., 10 seconds at 52° C., and 8 seconds at 72° C. (temperature ramp 20° C./second). FuT8 cDNA levels were normalized to those of the housekeeping gene ALAS using LightCycler Relative Quantification Software. Results are shown in the following Table 1.

TABLE 1
Light Cycler RT-PCR analysis of FuT8 mRNA expression.

| Clone: | % FuT8-expression mRNA |
|---|---|
| wild-type | 100 (reference) |
| Control shRNA | 69 |
| LCA, clone 1 | 2 |
| LCA, clone 2 | 34 |
| LCA, clone 3 | 16 |
| LCA, clone 4 | 23 |
| LCA, clone 5 | 6 |
| LCA, clone 6 | 5 |
| LCA, clone 7 | 15 |
| LCA, clone 8 | 127 |
| LCA, clone 9 | 3 |
| LCA, clone 10 | 23 |
| LCA, clone 11 | 14 |

LCA, clone 1 and LCA, clone 9 express a residual amount of α1,6-fucosyltransferase mRNA which is about 50- to 40-fold decreased.

### Example 4

### Mass Spectrometry Analysis of Antibody Glycostructure

The relative contents of sugar chain isoforms at Asn297 and Asn298, respectively, of the antibody were determined in glycosylated, intact antibody heavy chain (HC) by mass spectrometry as described in the following:

A) Purification of Antibody from Culture Supernatant of Cells Expressing Antibody and FuT8 shRNA

About 5-10 ml culture supernatants containing antibody (conc. ˜5-20 μg/ml) produced by cells also expressing shRNA against FuT8 were incubated with about 100 μl of a suspension of protein A-Sepharose™ CL-4B (30 mg/100 μl; Amersham Pharmacia Biotech AB) at 4° C. over night while inverting the vials. Afterwards, the samples were centrifuged in an Eppendorf centrifuge 5810R for 15 min. at about 400×g to sediment the protein A-Sepharose to which the antibody is bound. The culture supernatants were completely removed and the pellets were washed three times with about 50 μl doubly distilled water. After the third wash the solution was completely removed, about 30-50 μl of a 100 mM citrate-buffer, pH 2.8, were added to the pellets, and incubated while shaking for 15 min. at room temperature in order to release the antibody bound to protein A. After incubation, the suspensions were centrifuged for 5 min. at 14,000 rpm in an Eppendorf centrifuge and the resulting supernatants were carefully removed. The protein A pellets were washed once by adding about 30-50 μl 100 mM citrate buffer, pH 2.8, shaking for about 15 min. at room temperature and spinning down the protein A-Sepharose by centrifugation for 5 min. at 14,000 rpm in an Eppendorf centrifuge. The supernatants were removed carefully and combined with the respective solutions of the first release step. The protein A pellets were discarded.

### B) Analysis of the Oligosaccharide Structure Isoforms by ESI-Mass Spectrometry

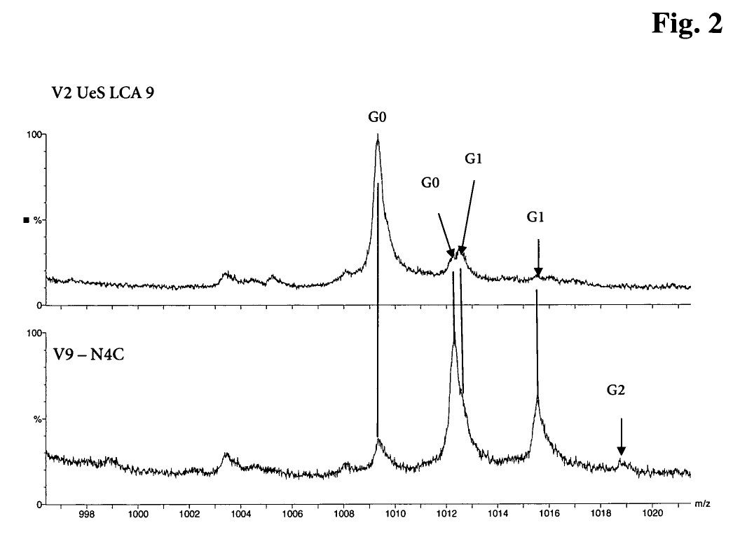

The antibody samples (˜60 μl, containing 20-50 μg each) obtained in step A) were denatured and reduced into light chain (LC) and glycosylated heavy chain (HC) by adding 100 μl 6 M guanidine-hydrochloride solution and 60 μl of a TCEP-guanidine-solution (1 M tris (2-carboxyethyl)-phosphine hydrochloride in 6 M guanidine-hydrochloride) to adjust the antibody solution to 3-4 M guanidine-hydrochloride and 250 mM TCEP. The sample was incubated for 1.5 h at 37° C. The reduced and denatured samples were desalted by G25 gel filtration with 2% formic acid (v/v) and 40% acetonitrile (v/v) as running buffer, and thereafter were subjected to offline, static ESI-MS analysis with nanospray needles (Proxeon Cat #ES 387) in a Q-Tof2- or a LCT-mass spectrometer instrument from Waters at a resolution of about 10000. The instrument was tuned according to manufacturer's instructions and calibrated with sodium iodine in a mass range from 500-2000 using a first order polynomial fit. Results are shown in FIG. 2.

During measurement of samples, routinely, 30-40 single scans in a mass range from 700-2000 were recorded and 10-30 single scans were added to yield the final m/z-spectrum used for evaluation.

Identification of the carbohydrate structures bound to the HC and calculation of the relative content of the individual sugar structure isoforms was done from the m/z spectra obtained. The deconvolution tool of the mass lynx software of waters was used to calculate the masses of the individual glycosylated HC-species detected. The respective carbohydrate structures attached to HC were assigned according by calculating the mass differences between the masses obtained for the individual glycosylated HC-species and the mass for non-glycosylated HC as deduced from the DNA sequence and by comparing these mass differences with theoretical masses of known N-linked glycol structures of antibodies.

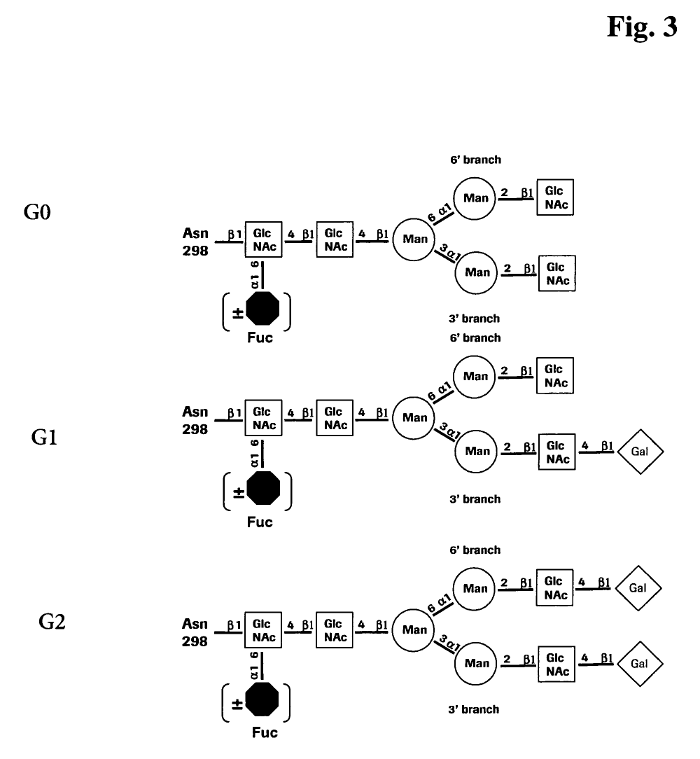

For determination of the ratios the oligosaccharide isoforms, the peak heights of the individual, differently glycosylated HC-species were determined from several selected single charge (m/z)-states, which do not overlap with other signals of other molecule species, like LC etc. For determination of the ratios the oligosaccharide isoforms, the peak heights of G0+Fuc and G0 (see FIG. 3) were determined from selected single charge (m/z)-states (an example see in FIG. 2). The relative content of sugar structures with different fucosylation, was deduced only from the ratio of the peak heights of the HC-species containing the G0-structure+fucose (G0+Fuc; complex, bi-antennary structure lacking terminal galactose residues and carrying core-fucosylation) and the HC-species containing the G0-structure−fucose (G0−Fuc; see FIG. 3a). For this determination the corresponding peaks within the same charge (m/z)-state were used (e.g. peaks of G0+fucose and G0 without fucose of m/z 45). Quantitative results are shown in Table 2.

TABLE 2
Percentage of fucosylation as determined by mass spectroscopy

| Clone | 100 - amount of fucosylation [%] |
|---|---|
| LCA, clone 1 | 88 |
| LCA, clone 9 | 83 |

### Example 5

### ADCC-Assay (Antibody Dependent Cellular Cytotoxicity)

The ADCC assay for the detection of tumor cell lysis induced by addition of an antibody produced by LCA clones 1 and 9 and a wild type cell as a control was performed according to the producer's manual (PerkinElmer, USA). As effector cells, freshly isolated peripheral blood cells were used, as target cells, DU145 cells were used. Results are shown in Table 3.

TABLE 3
ADCC Assay showing the percentage of released cells relative to 0.5%
Triton-treated cells (100% release).

| | % release, | | |
|---|---|---|---|
| | relative | % release, relative | % release, relative |
| ng/ml | to 0.5% Triton | to 0.5% Triton | to 0.5% Triton |
| antibody: | LCA, clone 1 | LCA, clone 9 | wild-type |
| 50 | 142.79 | 141.33 | 10.36 |
| 25 | 133.13 | 136.01 | 14.35 |
| 12.5 | 135.57 | 123.93 | 12.27 |
| 6.25 | 104.8 | 105.99 | 0.53 |
| 3.125 | 86.16 | 98.47 | 11.60 |
| 1.5625 | 61.82 | 55.3 | 14.07 |
| 0.78125 | 39.93 | 38.53 | 5.07 |
| 0.390625 | 14.11 | 20.2 | 5.77 |

### Example 6

### Stability of Silencing Effect

CHO-DG44/wild-type and CHO-DG44/LCA-clone 9 have been cultured for four weeks without selection pressure. Every week 1×10⁶ cells were plated on a 6 cm culture dish. 24 hrs later, cells were harvested. RNA isolation, cDNA-synthesis, quantitative RT-PCR and data analysis were performed as in Example 3. Results are shown in Table 4.

TABLE 4
Stability of silencing effect

| Clone/week in which | % FuT8 mRNA |
|---|---|
| cells were harvested | expression |
| wild-type | 100 (reference) |
| LCA, clone 9, week 1 | 8 |
| LCA, clone 9, week 2 | 9 |
| LCA, clone 9, week 3 | 9 |
| LCA, clone 9, week 4 | 9 |

1. A method for recombinantly producing a heterologous polypeptide with a reduced degree of fucose modification in a mammalian cell comprising the following steps:
cultivating said mammalian cell under conditions suitable for the expression of said heterologous polypeptide,
recovering the heterologous polypeptide from the mammalian cell or the culture and thereby producing said heterologous polypeptide,
wherein said mammalian cell is transfected with
i) a first nucleic acid of SEQ ID NO: 5 or of SEQ ID NO: 6 that is transcribed to an shRNA directed against α1,6-fucosyltransferase mRNA, and
ii) a second nucleic acid encoding a heterologous immunoglobulin, an immunoglobulin fragment, or an immunoglobulin conjugate.

2. The method according to claim 1, wherein step a) of cultivating said mammalian cells is in the presence of Lens culinaris agglutinin (LCA).

3. The method claim 2, wherein said mammalian cell is transfected with
iii) a third nucleic acid encoding a neomycin selection marker or I-NGFR.

4. The method of claim 3, wherein said mammalian cell is transfected with a single nucleic acid comprising
a first nucleic acid of SEQ ID NO: 5 or of SEQ ID NO: 6 that is transcribed to an shRNA directed against α1,6-fucosyltransferase mRNA,
a second nucleic acid encoding a neomycin selection marker or I-NGFR, and
a third nucleic acid encoding said heterologous polypeptide.

5. The method of claim 1, wherein said mammalian cell is selected from the group of mammalian cells comprising CHO cells, BHK cells, NS0 cells, SP2/0 cells, HEK 293 cells, HEK 293 EBNA cells, PER.C6 cells, and COS cells.

6. A nucleic acid comprising
a first nucleic acid selected from the group of nucleic acids of SEQ ID NO: 5 and 6,
a second nucleic acid encoding a neomycin selection marker or I-NGFR, and
a third nucleic acid encoding a heterologous polypeptide selected from the group of heterologous polypeptides comprising immunoglobulins, immunoglobulin fragments, and immunoglobulin conjugates.

7. A mammalian cell comprising the nucleic acid according to claim 6.
